(19) United States
(12) Patent Application Publication
(10) Pub. No.: US 2026/0224899 A1
(43) Pub. Date: Aug. 6, 2026

(54) SYSTEMS FOR PACING RATE PROGRAMMING

(71) Applicant: BAROPACE, INC., Ashland, OR (US)

(72) Inventors: Michael BURNAM, Ashland, OR (US); Srikanth JADCHERLA, Frisco, TX (US); Taral OZA, San Jose, CA (US)

(73) Assignee: BAROPACE, INC., Ashland, OR (US)

(21) Appl. No.: 19/152,134

(22) PCT Filed: Jan. 26, 2024
(86) PCT No.: PCT/US2024/013184

§ 371 Date: Jul. 30, 2025

Related U.S. Application Data
(60) Provisional application No. 63/482,254, filed on Jan. 30, 2023.
(60) Provisional application No. 63/595,097, filed on Nov. 1, 2023.

(51) Int. Cl. A61N 1/365; A61N 1/372

(52) U.S. Cl. CPC A61N 1/36585; A61N 1/37247

(57) ABSTRACT

Disclosed are methods and systems for outputting a pacing rate, including: receiving a heart rate and a blood pressure of a patient; determining a first pacing rate to modify the heart rate based on the heart rate and the blood pressure; outputting the first pacing rate; receiving a subjective input subsequent to outputting the first pacing rate; determining a second pacing rate based on the subjective input; and outputting the second pacing rate.

### CROSS-REFERENCE TO RELATED APPLICATIONS

This application claims priority to U.S. Provisional Application No. 63/482,254 filed Jan. 30, 2023 and U.S. Provisional Application No. 63/595,097 filed Nov. 1, 2023, the entirety of each of which is incorporated by reference herein.

### TECHNICAL FIELD

Various embodiments of the present disclosure relate generally to cardiac pacing based on at least one input, and more particularly, to systems and methods for outputting cardiac pacing rates based on one or more objective and/or subjective inputs.

### INTRODUCTION

Hypertension (HTN) is a major contributor to cardiovascular mortality. Many patients with drug-resistant hypertension (DRH) also require permanent pacing (PP). Hypertension treatment with a dual-chamber pacemaker appears safe and effective at intermediate and long-term follow-up.

Generally, a pacing rate for a pacemaker may be determined by an internal algorithm of the pacemaker. The base rate of the pacemaker, or the lowest heart rate allowed, may be set by a cardiologist taking into account the output of rhythm studies and other tests. Current approaches to treatment rely on a patient's presence at a provider's to allow of pacing rate adjustment.

Unless otherwise indicated herein, the materials described in this section are not prior art to the claims in this application and are not admitted to be prior art, or suggestions of the prior art, by inclusion in this section.

### SUMMARY OF THE DISCLOSURE

Various embodiments of the present disclosure relate generally to methods and systems for pacing rate programming.

In one aspect, an exemplary embodiment of methods and systems for outputting a pacing rate may include: receiving a heart rate and a blood pressure of a patient; determining a first pacing rate to modify the heart rate based on the heart rate and the blood pressure; outputting the first pacing rate; receiving a subjective input subsequent to outputting the first pacing rate; determining a second pacing rate based on the subjective input; and outputting the second pacing rate.

Determining the first pacing rate to modify the heart rate may further be based on at least one of physiological parameters, environmental parameters, and/or emotional parameters. The physiological parameters include one or more of the patient's current heart rate, current blood pressure, temperature, medication compliance, blood oxygen level, glucose level, blood electrolytes level, an accelerometer value, a respiratory rate sensor value, a thoracic impedance, an impedance, portions of cardiac rate such as atrial rate, ventricular rate, atrioventricular conduction, rhythm irregularities, autonomic nervous system (ANS) function, glucose, skin electrolytes, galvanic skin response, Photoplethysmography (PPG) values, electroencephalogram (EEG) wave, and/or urination parameters. The environmental parameters may include one or more of a patient diet, a time of day, an ambient temperature, a patient location, an ambient oxygen concentration, or a humidity. The first pacing rate or the second pacing rate may be output by a pacing machine learning algorithm. The blood pressure may be systolic or diastolic.

This aspect may include determining whether the blood pressure of the patient is one of above a first threshold blood pressure or below a second threshold blood pressure; and determining the first pacing rate further based on whether the blood pressure of the patient is one of above the first threshold blood pressure or below the second threshold blood pressure. The first pacing rate may be one of higher than the heart rate by a first pacing value if the blood pressure of the patient exceeds the first threshold blood pressure or is lower than the heart rate by a second pacing value different than the first pacing value, if the blood pressure of the patient is below the second threshold blood pressure.

This aspect may include, upon expiration of a predetermined time period or a dynamically determined time period: receiving an updated heart rate and an updated blood pressure of the patient; determining a third pacing rate to modify the updated heart rate based on the updated heart rate and the updated blood pressure; outputting the third pacing rate; receiving an updated subjective input subsequent to outputting the third pacing rate; determining a fourth pacing rate based on the updated subjective input and the third pacing rate; and outputting the fourth pacing rate. The first and second pacing rates may be further determined based on patient attributes. Receiving the subjective input may include: receiving a sensor input from a physiological sensor; providing the sensor input to a pseudo-subjective machine learning model trained to generate outputs based on at least one of historical sensor inputs, simulated sensor inputs, historical subjective inputs, or simulated subjective inputs; and receiving, as an output from the pseudo-subjective machine learning model, the subjective input. The first pacing rate may be further based on one of a blood pressure device, a pacing device, or a subjective input device.

In another aspect, a system for outputting a pacing rate may include: a processor; and a memory having programming instructions stored thereon, which, when executed by the processor, causes the system to perform operations comprising: receiving a heart rate and a blood pressure of a patient; determining a first pacing rate to modify the heart rate based on the heart rate and the blood pressure; outputting the first pacing rate; receiving a subjective input subsequent to outputting the first pacing rate; determining a second pacing rate based on the subjective input; and outputting the second pacing rate.

Determining the first pacing rate to modify the heart rate may be further based on at least one of physiological, environmental, and/or emotional parameters. The operations may further include: determining whether the blood pressure of the patient is one of above a first threshold blood pressure or below a second threshold blood pressure; determining the first pacing rate further based on whether the blood pressure of the patient is one of above the first threshold blood pressure or below the second threshold blood pressure. Receiving the subjective input may include receiving a sensor input from a physiological sensor; providing the sensor input to a pseudo-subjective machine learning model trained to output the subjective input based on at least one of historical sensor inputs, simulated sensor inputs, historical subjective inputs, or simulated subjective inputs; and receiving, from the pseudo-subjective machine learning model, the subjective input.

According to another aspect, a non-transitory medium for outputting a pacing rate and having a sequence of instructions, which, when executed by a processor, causes a computing system to perform operations may include: receiving a heart rate and a blood pressure of a patient; determining a first pacing rate to modify the heart rate based on the heart rate and the blood pressure; outputting the first pacing rate; receiving a subjective input subsequent to outputting the first pacing rate; determining a second pacing rate based on the subjective input; and outputting the second pacing rate.

Determining the first pacing rate to modify the heart rate may be further based on at least one of physiological, environmental, and/or emotional parameters. This aspect may further include determining whether the blood pressure of the patient is one of above a first threshold blood pressure or below a second threshold blood pressure; and determining the first pacing rate further based on whether the blood pressure of the patient is one of above the first threshold blood pressure or below the second threshold blood pressure. Receiving the subjective input may include: receiving a sensor input from a physiological sensor; providing the sensor input to a pseudo-subjective machine learning model trained to output the subjective input based on at least one of historical sensor inputs, simulated sensor inputs, historical subjective inputs, or simulated subjective inputs; and receiving, from the pseudo-subjective machine learning model, the subjective input.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. Moreover, there are many embodiments described and illustrated herein.

FIG. 1 depicts an exemplary environment for determining a pacing rate, according to one or more embodiments.

FIG. 2A depicts a flow diagram of an exemplary method for determining a pacing rate, according to one or more embodiments.

FIG. 2B depicts a lookup table, according to one or more embodiments.

FIG. 3 depicts a flowchart of an exemplary method for determining a pacing rate, according to one or more embodiments.

FIG. 4 depicts a further flow diagram of an exemplary method for determining a pacing rate via a pseudo-subjective machine learning model, according to one or more embodiments.

FIG. 5 depicts differences of mean systolic and diastolic blood pressures prior to and after cardiac pacing in responder and non-responder patient groups, according to one or more embodiments.

FIGS. 6A and 6B illustrate the distribution of systolic blood pressure values before and after cardiac pacing, according to one or more embodiments.

FIG. 7 depicts the relationship between cardiac pacing, decline in systolic blood pressure, and changes in medications, according to one or more embodiments.

FIG. 8 depicts the distribution of atrial pacing among patients, according to one or more embodiments.

FIG. 9A depicts the increase in exercise duration in patient groups, according to one or more embodiments.

FIG. 9B depicts the increase in systolic blood pressure of a patient group, according to one or more embodiments.

FIG. 10 depicts an example of training a machine learning model, according to one or more embodiments.

FIG. 11 depicts an example of a computing device, according to one or more embodiments.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION OF EMBODIMENTS

Various embodiments of the present disclosure relate generally to methods and systems for cardiac pacing rate programming

Reference will now be made in detail to examples of the present disclosure. The present disclosure is not limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Notably, an embodiment or implementation described herein as “exemplary” is not to be construed as preferred or advantageous, for example, over other embodiments or implementations; rather, it is intended to reflect or indicate the embodiment(s) is/are “example” embodiment(s). The term “exemplary” is used in the sense of “example” rather than “ideal.”

Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

In this disclosure, the term “based on” means “based at least in part on.” The singular forms “a.” “an.” and “the” include plural referents unless the context dictates otherwise.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus.

The term “or” is used disjunctively, such that “at least one of A or B” includes, (A), (B), (A and A), (A and B), etc. Relative terms, such as, “substantially,” “approximately,” and “generally,” are used to indicate a possible variation of 10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact. In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a user. e.g., to provide medical care, medical intervention or advice, or the like. Terms like “user,” “patient,” or the like generally encompass any person (e.g., an individual, a medical provider, etc.) or entity who is using a device, programming a device, obtaining information, seeking resolution of an issue, or the like.

Terms like “pacemaker” or the like generally encompass a device that may help control a user's heartbeat, for example, to prevent the user's heart from beating too fast or too slow. A pacemaker may include one or more sensors and/or one or more defibrillators, which may determine heart rate and provide electrical impulses, respectively. Terms like “pacing rate” or the like generally encompass the electrical pulses generated by the pacemaker and provided to one or more chambers of the heart to maintain an adequate heart rate. Terms like “base rate” or the like generally encompass the lowest heart rate allowed by a pacemaker. Terms like “lookup table” or the like generally encompass data that may determine the pacing rate and/or the base rate. A lookup table may be stored, e.g., in a database, in the form of a data table.

According to implementations of the disclosed subject matter, changes to a pacing rate and/or a base rate may be based on a lookup table. A lookup table may be a table with the patient attributes (e.g., height, weight, gender, medical condition, physiologic input, current state, etc.), objective inputs, subjective inputs, and/or the like and may be referenced to determine one or more pacing rates. Such a lookup table may be accessed by a pacemaker or a processing device in communication with a pacemaker.

According to implementations of the disclosed subject matter, cardiac pacing may be determined based on physiological inputs such as, but not limited to, blood pressure and/or heart rate, as further discussed herein. Such physiological input based cardiac pacing may be used to treat conditions such as, but not limited to, drug resistant hypertension (DRH), DRH with diastolic congestive heart failure (DCHF), heart failure with preserved ejection fraction (HFpEF), etc.

Blood pressure may be detected using a blood pressure measuring device (a “device” or a “blood pressure device”). A blood pressure may be a sensed value, a blood pressure, a sensed value converted into one or more other formats (e.g., by a processor), or the like. A blood pressure may indicate how much pressure a user's blood exerts against the user's artery walls when the user's heart beats (e.g., a systolic blood pressure). A blood pressure may indicate how much pressure a user's blood exerts against the user's artery walls when the user's heart is resting between beats (e.g., diastolic blood pressure).

A blood pressure measuring device may include any type of blood pressure monitor or cuff such as, for example, a pneumatic cuff relying on mechanical compression of a peripheral artery cuff (e.g., to be attached to brachial artery, ankle, wrist, etc.), a nonpneumatic cuff (e.g., which analyzes an arterial waveform and function anywhere on the body where the arterial pulse contour can be sensed such as at a wrist), or an implantable sensor within a blood vessel or heart chamber. A blood pressure measuring device may be a light-based device such as a photoplethysmography (PPG) device. A blood pressure measuring device may output blood pressure in a first format which may be converted to a second format such that a processing component receiving blood pressure information may be configured to utilize such information in the second format and may not be configured to utilize such information in the first format.

Physiological inputs, as discussed herein, include, but are not limited to, a blood-pressure, heart rate, biomarker level (e.g., cortisol, atrial natriuretic peptide (ANP). B-type natriuretic peptide (BNP), N-terminal pro b-type natriuretic peptide (NT-proBNP), etc.), blood oxygen level, glucose level, blood electrolytes level, an accelerometer value, respiratory rate sensor value (e.g., via diaphragmatic movement), thoracic impedance, impedance (e.g., as a correlate of right ventricular function), environmental parameter, ambient oxygen concentration (e.g., SPO2), humidity, portions of cardiac rate such as atrial rate, ventricular rate, atrioventricular conduction, the presence of rhythm irregularities, autonomic nervous system (ANS) function, glucose, skin electrolytes, galvanic skin response, PPG values, Electroencephalogram (EEG) wave, urination parameters, etc. Such physiological inputs may be provided by one or more sensors, devices, or the like. Systolic blood pressure (SBP) and diastolic blood pressure (DBP) may be sensed by one or more blood pressure sensing devices.

According to implementations of the disclosed subject matter, cardiac pacing may be determined based on environmental parameters. Such environmental parameters include one or more of the patient's diet, a time of day, an ambient temperature, the patient's location, an ambient oxygen concentration, and/or a humidity.

Additionally, cardiac pacing may be determined based on subjective input from the patient. This subjective input can include emotional parameters, such as the patient's (or a patient's caregiver's) reporting of a state of emotional well-being, physical well-being, comfort level, etc.

As discussed herein, cardiac pacing may be based on physiological parameters, environmental parameters, and/or subjective/emotional parameters. It will be understood that such parameters include changes to such parameters. For example, cardiac pacing may be based one or more of a change such as a change in clinical status, a change in medication, a change in other physiologic parameters, a change in other diagnostic testing such as in vitro diagnostics (e.g., blood tests and the like), changes based on procedures during and/or after surgery, endoscopy, cardiac ablation, renal denervation, etc.

Accordingly, techniques disclosed herein may be implemented to modify pacing (e.g., by a pacing device such as a pacemaker) based on physiological, environmental, and/or subjective inputs. Pacing may be modified in accordance with an algorithm or machine learning output. For example, pacing may be modified to improve a blood pressure related condition by increasing or decreasing blood pressure, via determined cardiac pacing outputs, based on observed biomarker levels. The modification may result an increase in a cardiac pacing rate or amplitude, a decrease in cardiac pacing rate or amplitude, an acceleration of a cardiac pacing rate, a deceleration of a cardiac pacing rate, and/or the like. Such modified pacing may, at least in part, improve a given medical conditions for a patient (e.g., a blood pressure condition). Such conditions may include, for example, hypertension, hypotension, DRH, DRH with diastolic congestive heart failure (DCHF), HFpEF, and/or the like.

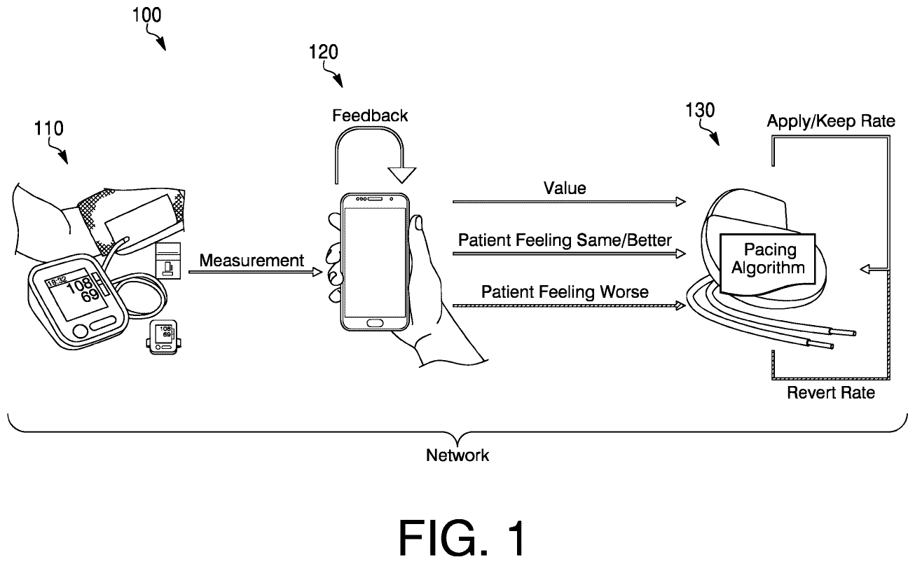

FIG. 1 shows an exemplary environment 100 for a closed-loop system for determining an adjusted physiologic pacing rate, according to one or more embodiments. Such a pacing rate may be determined, for example, upon detection of a medical condition such as Hypertensive Heart Disease and/or may be determined independent of a medical condition (e.g., based on one or more physiological inputs, subjective inputs, environmental inputs, and/or the like). Physiological pacing rates may include any applicable properties for cardiac pacing such as, but not limited to, frequency of pacing, amplitude of pacing, duration of pacing, acceleration of pacing, deceleration of pacing, etc. Environment 100 of FIG. 1 depicts at least one blood pressure measuring device 110, a component 120 to receive the input (e.g., an electronic device, a patient device, a network device, a cloud device, etc.), and pacing system 130. Some or all of these components may be connected via a network 140.

One or more of the components of environment 100 of FIG. 1 may communicate with each other and/or other systems, e.g., via network 140. In some embodiments, network 140 may connect one or more components of environment 100 via a wired connection. In some embodiments, network 140 may connect one or more aspects of environment 100 via an electronic network connection, for example a wide area network (WAN), a local area network (LAN), personal area network (PAN), or the like. In some embodiments, the electronic network connection includes the internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like. In any case, the connections within the environment 100 may be network, wired, any other suitable connection, or any combination thereof.

Although depicted as separate components in FIG. 1, it should be understood that a component or portion of a component in the environment 100 may, in some embodiments, be integrated with or incorporated into one or more other components. For example, physiological input measuring device 110 may be integrated with pacing system 130. In another example, pacing system 130 may further include at least one other physiological sensor. In some embodiments, operations or aspects of one or more of the components discussed above may be distributed amongst one or more other components. In some embodiments, some of the components of environment 100 may be associated with a common entity, while others may be associated with a disparate entity. Any suitable arrangement and/or integration of the various systems and devices of the environment 100 may be used.

Physiological input measuring device 110 may include one or more sensors such as, but not limited to, a blood-pressure sensors, heart rate sensors, biomarker level sensors, blood oxygen level sensors, glucose level sensors, blood electrolytes level sensors, an accelerometer, motion sensors, position sensors, respiratory rate sensors, impedance sensors, environmental sensors, ambient oxygen condition sensors, humidity sensors, ANS sensors, glucose sensors, skin electrolytes sensors, galvanic skin sensors, PPG sensors, EEG sensors, EKG sensors, fluid sensors, volume sensors, light sensors, cameras, and/or the like.

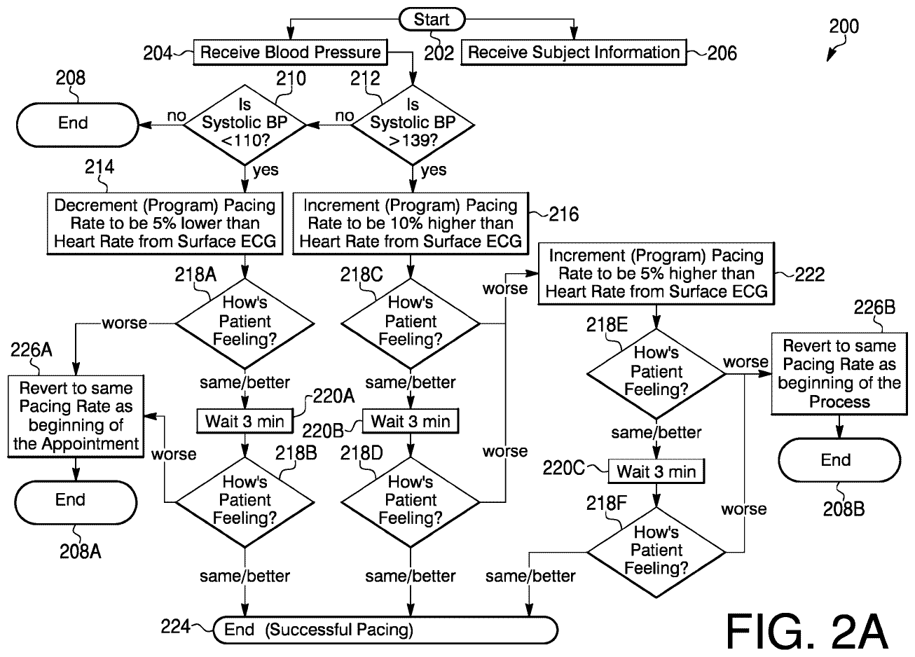

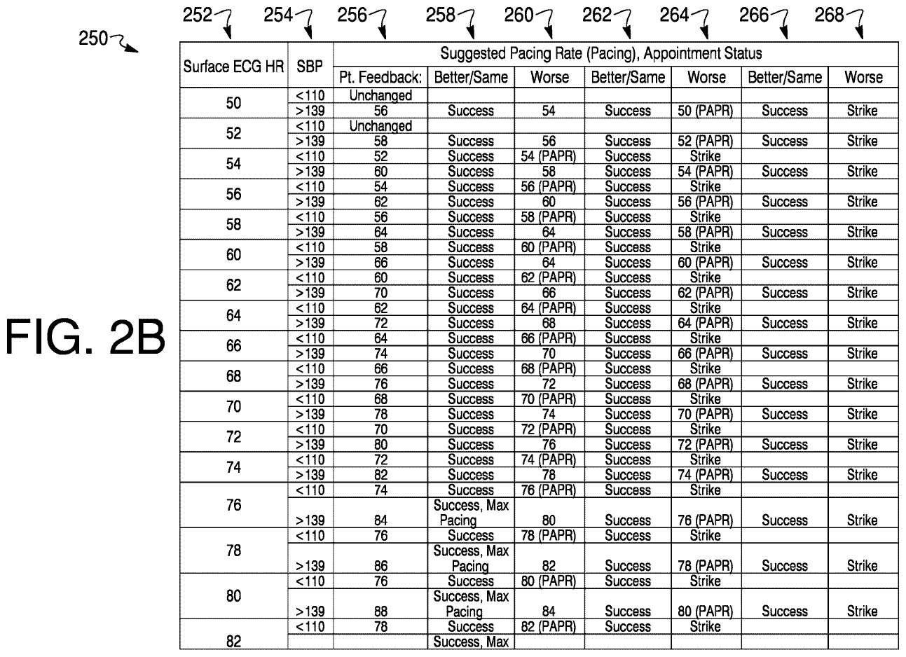

According to implementations of the disclosed subject matter, one or more systems or methods disclosed herein may be utilized for pacing rate programming. FIG. 2 depicts a flow diagram 200 determine (e.g., program) one or more cardiac pacing rates. It will be understood that the steps described in reference to FIG. 2A are an example only. Steps shown in FIG. 2A may be performed in the order described herein or in any applicable order. Further, for simplicity, flow diagram 200 is provided in reference to blood pressure data. However, it will be understood that any physiologic input (e.g., as discussed herein) may be substituted for blood pressure in accordance with the techniques discussed in reference to flow diagram 200.

As shown in FIG. 2A, the process of flow diagram 200 may start at step 202. Physiologic property data (e.g., blood pressure data) of a subject may be received at step 204. For example, blood pressure data may be received via physiological input measuring device 110 based on one or more sensor inputs detected at physiological input measuring device 110. Physiological property data, at step 204, may be received at, for example, component 120 via network 140. As discussed herein, physiological property data received at step 204 may be determined in a first format associated with physiological input measuring device 110 and may be converted into a second format to be utilized by component 120. Physiological property data received at step 204 may be converted from the first format to the second format via physiological input measuring device 110 and/or via component 120. According to an implementation, component 120 may be part of, associated with, or in communication with pacing system 130.

At step 206, subject information of a given subject may be received. Such subject information may include, for example, patient attributes (e.g., demographic attributes, height, weight, ethnicity, medical conditions, medication information, etc.). Subject information may be provided via physiological input measuring device 110 (which may be the same as or different than the physiological input measuring device that provides blood pressure information at step 204). Alternatively, or in addition, subject information may be provided via component 120 (e.g., via user input or storage associated with component 120) and/or may be provided via a separate component (e.g., a remote component, database, server, electronic medical record program, etc.).

At step 212, a determination may be made regarding whether a physiologic property (e.g., SBP) associated with the input received at step 204 meets or exceeds a first threshold physiologic value (e.g., if SBP is greater than approximately 139). The first threshold physiologic value may be an upper bound of an acceptable physiologic value range (e.g., an acceptable SBP range). If the physiologic property does not meet or exceed the first threshold physiologic value, then step 210 may be performed. At step 210, a determination may be made whether the physiologic property is below a second threshold physiologic value (e.g., if SBP is less than approximately 110). The second threshold physiologic value may be a lower bound of an acceptable physiologic value range (e.g., an acceptable SBP range). If the physiologic property is not below the second threshold physiologic value, then step 208 may be performed and the process of flow diagram 200 may be terminated. Accordingly, step 208 may be performed to terminate the process if the physiologic property (e.g., blood pressure) received at step 204 is within an acceptable physiologic value range. For example, the physiologic property data being in such an acceptable range may not require an adjusted pacing rate.

Returning to step 210, if the physiologic property is below the second threshold physiologic value (e.g., lower than the acceptable physiologic value range), step 214 may be performed. For example, as shown in flow diagram 200, if systolic blood pressure is less than approximately 110, an existing pacing rate may be adjusted to be a predetermined or dynamically determined amount (e.g., approximately 5%) lower. The pacing rate may be adjusted to be lowered by the amount in comparison to the existing pacing rate and/or may be adjusted to be lowered in comparison to a heart rate (e.g., a heart rate detected by an ECG sensor).

According to an embodiment, the pacing rate may be adjusted based on an output of a pacing machine learning model. The pacing machine learning model may be trained in accordance with the techniques disclosed herein. For example, the pacing machine learning model may be trained to adjust one or more weights, layers, biases, synapses, nodes, and/or the like based on training data that may include one or more of historical or simulated physiologic input values, subject information, acceptable physiologic value ranges, pacing rates, pacing rate changes, subjective inputs, and/or the like. The pacing machine learning model may receive, as inputs, one or more of the physiologic input value received at step 204, the subject information received at step 206, the acceptable physiologic value range applied at steps 212 and 210 (e.g., as output by a threshold machine learning model), and/or the like. The pacing machine learning model may output a pacing rate (e.g., an actual rate, a percentage change, a ratio, etc.) based on the inputs.

Next, at step 218A, a subjective input may be received from the subject. For example, a patient may be provided a prompt (e.g., via a graphical user interface) requesting the patient to provide an assessment of his or her own wellbeing. According to an implementation of the disclosed subject matter, the subject may be provided a prompt, via a graphical interface, comprising graphical components generated based on one or more of the physiologic property received or determined based on the input received at step 204, based on the amount of deviation of the physiological property from the second threshold physiologic value, and/or the like. For example, the prompt may include larger icons for receiving the subjective input if the amount of deviation of the physiological property from the second threshold physiologic value is above a threshold amount. An order of the requested input may be determined based on the one or more of the physiologic property received or determined based on the input received at step 204, based on the amount of deviation of the physiological property from the second threshold physiologic value, and/or the like. For example, a graphical component corresponding to the subject feeling ill may be ordered above a graphical component corresponding to the subject feeling healthy if the amount of deviation of the physiological property from the second threshold physiologic value is above a threshold amount.

If, at step 218A, a positive indication is received indicating that the subject is feeling the same or better than prior to the pacing rate adjustment at step 214, a waiting period of a predetermined or dynamically determined time period (e.g., 3 minutes) may be implemented at step 220A. Upon expiration of the time period, another subjective input may be received from the subject at 218B. According to implementations of the disclosed subject matter, a dynamically determined time period may be determined using an algorithm or time-based machine learning model. The time-based machine learning model may be trained in accordance with the techniques disclosed herein. For example, the time-based machine learning model may be trained to adjust one or more weights, layers, biases, synapses, nodes, and/or the like based on training data that may include one or more of historical or simulated physiologic input values, subject information, acceptable physiologic value ranges, and/or the like. The time-based machine learning model may receive, as inputs, one or more of the physiologic input value received at step 204, the subject information received at step 206, the acceptable physiologic value range applied at steps 212 and 210, and/or the like. The time-based machine learning model may output a dynamically determined time period such that, for example, the dynamically determined time period provides sufficient time for the subject to notice an effect of the pacing rate change implemented at step 214. Further, the output dynamically determined time period may not exceed a time period such that the likelihood of the subject being effected by external factors other than the pacing rate change is below an acceptable threshold likelihood. Accordingly, the time-based machine learning model may, at least in part, determine a likelihood of the subject being effected by external factors.

If, at step 218B, a positive indication is received indicating that the subject is feeling better or the same as prior to step 220A, the process may be considered successful and may terminate at step 224. It will be understood that indications (e.g., via subjective inputs received at steps 218A, 218B, 218C, 218D, 218E, and/or 218F) may be provided via an interface in a manner similar to that discussed in reference to step 218A.

If at step 218A, a negative indication is received indicating that the subject is feeling worse, then, at step 226A, the pacing rate will revert to the same pacing rate at step 202, and the appointment will end 208A. Alternatively, at step 226A, the pacing rate may be adjusted to an intermediate rate (e.g., a rate higher than the pacing rate at step 202 but lower than the pacing rate at step 222).

Similarly, if at step 218B a negative indication is received indicating that the subject is feeling worse, then the pacing rate will revert to the same pacing rate at step 202, and the appointment will end 208A. Alternatively, at step 226B, the pacing rate may be adjusted to an intermediate rate (e.g., a rate higher than the pacing rate at step 202 but lower than the pacing rate at step 222

Still referring to FIG. 2A, if the physiological property value (e.g., systolic blood pressure) at step 212 is greater than the first threshold amount (e.g., approximately 139), the pacing rate may be adjusted to be raised by a predetermined or dynamically determined amount (e.g., 10%) at step 216. The pacing rate may be adjusted by the amount in comparison to the existing pacing rate and/or may be adjusted to be lowered in comparison to a heart rate (e.g., a heart rate detected by an ECG sensor).

Next, at step 218C, a subjective input may be received from the subject. If, at step 218C, a positive indication is received indicating that the subject is feeling the same or better than prior to the pacing rate adjustment at step 216, a waiting period of a predetermined or dynamically determined time period (e.g., approximately 3 minutes) may be implemented at step 220B. Upon expiration of the time period, another subjective input may be received from the subject at 218D.

If, at step 218D, a positive indication is received indicating that the subject is feeling better or the same as prior to step 220B, the process may be considered successful and may terminate at step 224. It will be understood that indications (e.g., via subjective inputs received at steps 218A, 218B, 218C. 218D, 218E, and/or 218F) may be provided via an interface in a manner similar to that discussed in reference to step 218A.

If at step 218C, a negative indication is received indicating that the subject is feeling worse, then, the pacing rate may be adjusted to be raised by a predetermined or dynamically determined amount (e.g., 5%) at step 222, as discussed herein. Similarly, if at step 218D a negative indication is received indicating that the subject is feeling worse, then the pacing rate may be adjusted to be raised by a predetermined or dynamically determined amount (e.g., 5%) at step 222, as discussed herein.

Next, at step 218E, a subjective input may be received from the subject. If, at step 218E, a positive indication is received indicating that the subject is feeling the same or better than prior to the pacing rate adjustment at step 222, a waiting period of a predetermined or dynamically determined time period (e.g., approximately 3 minutes) may be implemented at step 220C. Upon expiration of the time period, another subjective input may be received from the subject at 218F. If, at step 218F, a positive indication is received indicating that the subject is feeling better or the same as prior to step 222, the process may be considered successful and may terminate at step 224.

If at step 218E, a negative indication is received indicating that the subject is feeling worse, then, at step 226B, the pacing rate will revert to the same pacing rate at step 202, and the process may terminate at step 208B. Alternatively, at step 226B, the pacing rate may be adjusted to be higher than the pacing rate at step 202 but lower than the pacing rate at step 222. Similarly, if at step 218F a negative indication is received indicating that the subject is feeling worse, then the pacing rate will revert to the same pacing rate at step 202, and the process may terminate at step 208B. Alternatively, at step 226B, the pacing rate may be adjusted to be higher than the pacing rate at step 202 but lower than the pacing rate at step 222.

As discussed herein, a dose-response relationship to modifying a pacing rate (e.g., an atrial pacing rate) is provided herein. Accordingly, a pacing rate output to a pacing system may be based on a current or prior pacing rate. The output pacing rate may be a percentage or ratio of the current or prior pacing rate. The percentage or ratio may be predetermined (e.g., approximately 5%, approximately 10%, etc.) and/or may be determined based on or more factors discussed herein (e.g., via an algorithm or a pacing machine learning algorithm). Accordingly, for a first patient, the output pacing rate may be a percentage or ratio that is different for a second patient. As another example, for a first patient, the output pacing rate may be a percentage or ratio at a first time that is different than the output pacing rate for the first patient at a second time (e.g., based on a change in the patient information, physiological factors, etc.) In some embodiments, the adjustment of the base pacing rate or the atrial pacing rate may be adjusted based on a patient's tolerance and/or may not exceed the pacing maximum allowed by physiologic or safety guardrails (e.g., approximately 88 bpm). Such physiologic or safety guardrails may be determined based on patient information, device characteristics, safety regulations, and/or the like.

According to embodiments of the disclosed subject matter, the first threshold and/or the second threshold (e.g., as described in reference to steps 212 and/or 210) may be output by a threshold machine learning model. The threshold machine learning model may be trained in accordance with the techniques disclosed herein. For example, the threshold machine learning model may be trained to adjust one or more weights, layers, biases, synapses, nodes, and/or the like based on training data that may include one or more of historical or simulated physiologic input values, subject information, acceptable physiologic value ranges, pacing rates, pacing rate changes, changes in physiological values, and/or the like. The threshold machine learning model may receive, as inputs, one or more of the physiologic input value received at step 204, the subject information received at step 206, and/or the like. The threshold machine learning model may output the first threshold and/or second threshold, either of which may be patient specific. According to an embodiment, an output of the threshold machine learning model may be provided as an input into the time-based machine learning model which may provide an output, as discussed herein, at least in part of the threshold machine learning model output.

According to an embodiment of the disclosed subject matter, a complex lookup table may be accessed to determine a pacing rate. A primary lookup variable for the complex lookup table may be one or more physiological parameter values (e.g., blood pressure values) which may include reference systolic/diastolic numbers associated with respective blood pressure readings. Each pair of systolic/diastolic numbers may correspond to an applicable pacing rate. Hence, in response to an input physiological parameter value (e.g., blood pressure reading), the device and/or algorithm implementing the change of pacing rate may perform a look up of the new pacing rate to be programmed. An implementation of the complex lookup table (lookup table) may include a function that retrieves data from a pre-programmed and/or pre-populated array of data. For example, such pre-programmed and/or pre-populated array of data may be populated using the threshold machine learning model discussed herein. According to this example, the threshold machine learning model may be provided physiological parameter values as an input, and may generate corresponding pacing rates to populate the lookup table. For example, blood pressure (BP) may be looked up and a pacing rate may be determined using a device, based on populated lookup table.

The exemplary lookup table, as shown in FIG. 2B as Table 250, may be populated based on a patient's profile (e.g., using the threshold machine learning model and/or pacing machine learning model). Table 250 may include a heart rate 252, physiological parameter thresholds 254 (e.g., SBP) which may be output by a threshold machine learning model, a first pacing rate 256 which may be output by a pacing machine learning model, a first subjective input 258, a second pacing rate 260 output by a pacing machine learning model, a second subjective input 262, a third pacing rate 264 output by a pacing machine learning model, a third subjective input 266, a fourth pacing rate 268 output by a pacing machine learning model, and/or the like. It will be understood that the columns of Table 250 may corresponds to the steps described in reference to FIG. 2A, which may be iterated multiple times. Such iterations are not limited by the columns of Table 250 shown in FIG. 2B.

In some embodiments, the lookup table may be populated based on and/or may include a patient's height, weight, demographics, disease quantification (e.g. biomarkers such as one or more QT interval and/or one or more QTc interval), etc. The lookup table can be generated, for example, either using an empirical series of measurements in the clinic and/or a series of past records of the patient. Accordingly, a lookup table may be generated, for example, empirically or based on past historical information.

In some embodiments, the lookup table may not be static. In accordance with the current disclosure, a feedback system may be maintained wherein, for each new pacing rate that is programmed, the resultant physiological parameter values may be measured and/or monitored. If the desired control is achieved, changes to the look up table may not be needed. If the desired control is not achieved, a new target pacing rate may be tried/tested (e.g., in accordance with flow diagram 200 of FIG. 2A). If this rate results in the desired control, this experience may be recorded. After a certain number of such experiences, which may be a parameterized number, the lookup table entry may be modified to reflect the experiences. As such, a lookup table may be developed for each patient. Accordingly, a lookup table may be personalized (e.g., over time) based on feedback and learning, as discussed herein.

In certain clinical situations, it may be necessary to review modifications to the lookup table, even if such changes are algorithmically implemented autonomously. Hence, implementations of the current disclosure may include a built-in alert and/or communication mechanism that may be configured to transmit the modifications to a provider and may affect the changes, once approved. A user (e.g., a physician) or automated system may select, for example, either a review-gated mode or autonomous mode for the lookup table modifications or may set a threshold beyond which a change needs to be reviewed. Accordingly, a review (e.g., a manual review) may be implemented and may be triggered based on one or more thresholds.

At a macro level, correlating the personalized tables over similar patients (e.g., similar in height, weight, demographics, disease conditions, etc.) may yield improved starting points for the lookup table of each patient and/or also lead to the modifications of the lookup tables across a cohort. Such cross communication and group-evolution of lookup tables may be accomplished across multiple patient devices through a set of cloud services, anonymously. Such cross communication may be done at any desired or determined periodic frequency. Accordingly, cohort level data may be improved by aggregating personal tables and/or personal tables may be improved using cohort level data.

In some instances, modifications to a lookup table may inadvertently result in the patient feeling worse. In this case, a previous lookup table may be restored. Accordingly, previous versions of lookup tables may be stored and any may be restored, e.g., as a roll back feature. Accordingly, previous versions of a lookup table may be restored or rolled back.

In some instances, the lookup table may be a substitute implementation used in compute-constrained and/or battery-constrained platforms. For such a platform, the outputs of an algorithm may be pre-computed and stored, e.g., as a cache. This configuration may reduce the need for computation, reduce latency, and/or improve both the thermal management as well as battery life of certain implementations. Accordingly, a complex algorithm (e.g., running in the background) may be used to process one or more scenarios (e.g., a series of scenarios) and corresponding outputs bay be pre-recorded.

While the disclosed methods, devices, and systems are described with exemplary reference to pacing rate programming via a pacemaker, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

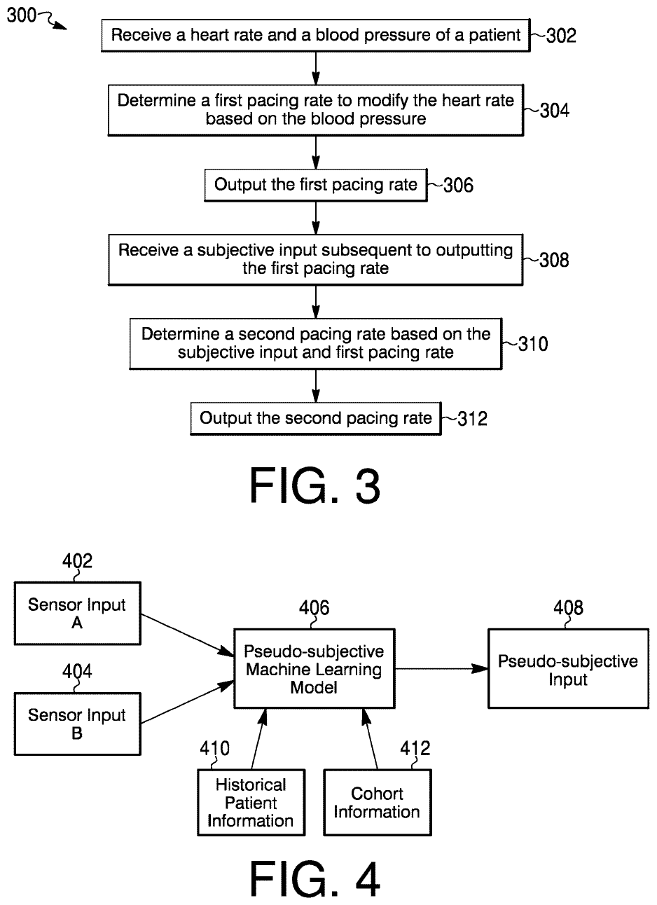

FIG. 3 shows a flowchart 300 for determining pacing rate, according to one or more embodiments. For simplicity, flowchart 300 is provided in reference to blood pressure. However, it will be understood that any applicable physiological parameter value, such as those discussed herein, may be used in reference to flowchart 300. At step 302, the beginning of a session, a patient's blood pressure and heart rate may be received. The blood pressure may be detected using a blood pressure cuff or a continuous blood pressure device. The blood pressure data may be provided to a processor that is local to a pacing device or a remote component external to the pacing device (e.g., a user device, a cloud component, an external processor, etc.), such as component 120 of FIG. 1.

At step 304, a first pacing rate may be determined to modify the heart rate received at step 302, based on the blood pressure input received at step 302. For example, if a patient's initial blood pressure was 145 mmHg and heart rate was 60 bpm, the first pacing rate may be set at 66 bpm. In another example, if a patient's initial blood pressure was 105 mmHg, and heart rate was 60 bpm, the first pacing rate may be set at 58 bpm. The first pacing rate may be determined based on the techniques disclosed herein in reference to FIG. 2A. For example, the first pacing rate may be determined based on a first threshold value and/or second threshold value output by a threshold machine learning model and/or an output generated by a pacing machine learning model, as discussed herein. Alternatively, or in addition, the first pacing rate may be determined in accordance with the complex lookup table discussed in reference to FIG. 2B. As discussed herein, the complex lookup table may be populated using outputs output by a threshold machine learning model and/or pacing machine learning model.

According to embodiments disclosed herein, a pacing rate may be determined on-demand using the threshold machine learning model and/or pacing machine learning machine learning model. For example, component 120 may receive the blood pressure and/or heart rate at step 302 and provide the same to the threshold machine learning model and/or pacing machine learning machine to receive a pacing rate. Alternatively, pacing system 130 may be provided a complex lookup table (e.g., table 250 of FIG. 2B) which may be populated based on the threshold machine learning model and/or pacing machine learning machine. Pacing system 130 may maintain a static copy of the complex lookup table which may be updated from time to time. According to this embodiment, battery efficiency may be optimized at the pacing system 130 by using the static complex lookup table to receive, determine, or otherwise identify pacing rates.

At step 306, the first pacing rate may be output and the output may be received at a cardiac pacing device (e.g., pacing system 130). The cardiac pacing device may be configured to pace based on the first pacing rate. For example, the first pacing rate may be output by a pacing machine learning algorithm and/or by a complex lookup table populated at least in part based on a pacing machine learning model.

After a defined period of time (e.g., as determined based on a time-based machine learning model), a patient may be provided an interface to rate their wellbeing (e.g., as discussed in reference to steps 218A, 218B, 218C, 218D, 218E, and/or 218E of FIG. 2A). The interface may be populated in accordance with the techniques discussed herein.

At step 308, a patient's input regarding their wellbeing may be received following the output of the first pacing rate at step 306. For example, a patient may indicate that they are feeling better, worse, or the same. If the patient is feeling better or same (e.g., based on a positive indication provided via the provided interface), the session may terminate as there may be no need for further adjustment of the pacing rate. However, if the patient indicates that they are feeling worse (e.g., a negative indication), the session may continue to step 310.

At step 310, a second pacing rate is determined based on input received from the patient at step 308. For example, if the first pacing rate was set at 66 bpm, the second pacing rate may be set at 63 bpm, based on input received from the patient at step 308. In another example, if the first pacing rate was set at 58 bpm, and the patient input a negative indication at step 308, the second pacing rate may be adjusted in accordance with the techniques disclosed herein (e.g., the second pacing rate may be set to the initial rate at the beginning of the session, such as 60 bpm). At step 312, the second pacing may be output and the output may be received at a cardiac pacing device. The cardiac pacing device may be configured to pace based on the second pacing rate.

The disclosed subject matter provides a closed-loop system for determining cardiac pacing rates (e.g., based on the patient's blood pressure, heart rate, and/or other physiological, environmental, subjective and/or pseudo-subjective inputs) and stimulating a heart based on the determined pacing rates (e.g., by stimulating atrial tissue).

As discussed herein, a cardiac rate may be determined at least in part based on a subjective input (e.g., received at steps 218A, 218B, 218C, 218D, 218E, and/or 218E of FIG. 2A, and/or step 308 of FIG. 3). According to embodiments of the disclosed subject matter, the subjective input may be a pseudo-subjective input. A pseudo-subjective input may be applied in place of the subjective input disused herein, and may be determined based on objective inputs.

As shown in FIG. 4, the pseudo-subjective input 408 be output by a pseudo-subjective machine learning model 406. The pseudo-subjective machine learning model 406 may be trained to output a pseudo-subjective input 408 based on one or more objective inputs. The pseudo-subjective machine learning model 406 may be trained by modifying one or more layers, weights, biases, synapsis, and/or the like of the model based on the training data. The machine learning model may be trained to output a pseudo-subjective input 408 based on one or more sensor inputs which may correspond to physiological inputs (e.g., blood pressure, heart-rate, temperature, sympathetic nerve activity (SNA), etc.). These sensor inputs may be sensed by one or more sensors, such as those shown in FIG. 4 as Sensor Input A 402 and/or Sensor Input B 404.

The pseudo-subjective machine learning model 406 may be trained using historical patient information 410, such as, patient subjective inputs, patient medications (e.g., patient provided, received by a system or component, medication compliance information, etc.), health history, and/or the like. For example, pseudo-subjective machine learning model 406 may be trained using historical subjective inputs provided by a patient (e.g., in accordance with flow diagram 200 of FIG. 2A). The pseudo-subjective machine learning model 406 may correlate or otherwise associate such subjective inputs with corresponding physiological inputs captured at or about the time the subjective inputs are provided. Accordingly, the pseudo-subjective machine learning model 406 may be trained to output pseudo-subjective inputs that correspond to the subjective inputs a patient is likely to provide, based on one or more current physiological states the patient is experiencing. As a simplified example, a given patient may typically provide a negative indication as a subjective input when the patient's heart rate is above a given amount. Accordingly, the pseudo-subjective machine learning model 406 may generate a negative indication pseudo-subjective input when sensor input A 402 and/or sensor input B 404 senses a heart rate above the given heart rate. It will be understood that although this simplified example is based on a single objective input (e.g., heart rate), the pseudo-subjective machine learning model 406 may be trained to output a pseudo-subjective input based on one or more objective inputs, trends related to such objective inputs, changes in such objective inputs, and/or the like.

Alternatively, or in addition, cohort population information 412 may be used to train pseudo-subjective machine learning model 406. For example, pseudo-subjective machine learning model 406 may be trained using historical or simulated subjective inputs provided by a cohort of patients (e.g., in accordance with flow diagram 200 of FIG. 2A). The pseudo-subjective machine learning model 406 may correlate or otherwise associate such cohort based subjective inputs with corresponding cohort physiological inputs captured at or about the time the subjective inputs are provided. Accordingly, the pseudo-subjective machine learning model 406 may be trained to output pseudo-subjective inputs that correspond to the subjective inputs a patient is likely to provide, based on one or more current physiological states the patient is experiencing. As a simplified example, a cohort of patients may typically provide a negative indication as a subjective input when the patients' temperature is above a given amount. Accordingly, the pseudo-subjective machine learning model 406 may generate a negative indication pseudo-subjective input when sensor input A 402 and/or sensor input B 404 senses that a given patient's temperature is above the given temperature.

### Experiment 1

The following disclosure corresponds to a first Experiment conducted in accordance with the subject matter discussed herein. Drug resistant hypertension (DRH) is defined as blood pressure (BP) that remains above goal despite concomitant use of ≥3 different classes of antihypertensive drugs, administered at maximally tolerated doses, including a diuretic. Patients with DRH are at high risk for having major cardiovascular events. The prevalence and incidence of DRH is expected to increase as the global population continues to age, with an overall increase in the number of affected individuals as the general population grows.

Recent efforts to address the problem of DRH have included the development and investigation of device-based therapies. Because many patients have high resting sympathetic activity, one focus in the past decade has been the development of several implantable devices, including those intended to target the autonomic nervous system, regulate left ventricular preload or alter mechanical arterial properties. Non-pharmacological neuromodulation devices that modulate sympathetic activity using electrical activation of the carotid baroreflex, catheter-based renal nerve ablation, and new algorithms for permanent ventricular pacing are supported by experimental studies and early clinical trials. However, the relationship between hypertension (HTN) and conventional clinical permanent cardiac pacing is not well established. Many older patients requiring permanent pacing (PP) also have persistent HTN with systolic blood pressures (SBP) above recommended levels. A significant reduction in SBP and diastolic (DBP) has been observed among such patients. In this experiment the effect of permanent cardiac pacing in a large group of elderly patients with DRH is evaluated and possible pathophysiological insights into pacing-mediated BP lowering is provided.

### Methods

Patient population: This Experiment includes a retrospective review of charts of patients who had undergone implantation of a permanent dual chamber pacemaker for standard clinical indications. Patients had documentation of DRH, e.g., hypertension requiring 3 or more antihypertension medications, with one of them usually being a diuretic. Patients with persistent atrial fibrillation were excluded from the study. Standard demographic data were collected, as was a pre- and post-implant left ventricular ejection fraction (LVEF). The patient's list of medications was reviewed and compared before and after the initiation of PP. The 6 months follow-up evaluation and resting BP cuff measurement at that visit were used for determining whether a patient was a “Responder” or a “Non-Responder” to PP (see below for definition). The BP and medication data from subsequent clinic visits were also collected, but not used in the analysis. The study was approved by the institutional review board. Written informed consent was obtained.

Definition: Patients were defined at the 6 months follow-up visit as Responders to PP if they manifested a SBP decline of ≥5 mmHg or had reduced by ≥1 their antihypertension medication regimen.

Statistical analyses: The demographic table is stratified by “Responders” and “Non-Responders” groups. For categorical variables, percentages are reported for each group, p-values of Fisher's Exact tests for comparison between response groups are reported. For continuous variables, mean and standard deviation are reported for each group; also, p-values of Wilcoxon Rank Sum tests for comparison between response groups are reported. P-value of the Wald test is reported to compare the proportion of the defined “Responders” (126/176) to 5%. P-value of paired t-test is reported to compare the number of baseline medications to the number of post-implant medications for the defined groups. A linear regression line is fitted in each scatter plot to show the correlation between ventricular pacing/atrial pacing and BPs (SBP and DBP) for the defined “Responder”. Wilcoxon Signed Rank tests are calculated to compare the BP change (between baseline and post-pacing) for the two response groups. For the relevant tests, a p-value of less than 0.01 was considered to be significant.

### Results

Description of patient study patients: As shown in Table 1 below, the demographic characteristic of the two groups, Responders and Non-Responders, were similar with respect to age, gender distribution, and pacing indications. As will be discussed in greater detail below, Responders tended to pace the atrium more than Non-Responders. Responders as a group had a higher pre-PP SBP. The distribution of HTN medications between the two groups was similar, with the exception of calcium channel blockers (CCB), which were more prevalent among the Responders. Patients who exhibited a longer follow up (as shown in Table 1) remained Responders or Non-Responders, e.g., unchanged from their group designation at six months.

TABLE 1

| | Responders | | Non-Responders | | |
|---|---|---|---|---|---|
| | n = 126 | | n = 50 | | P-value |
| Age (years), mean (±SD) | 75.2 | (6.4) | 73.7 | (8.6) | 0.7 |
| Men, % | 76.2 | | 82 | | 0.5 |
| Pacing indication SSS, % | 61.9 | | 46 | | 0.06 |
| Pacing indication AVB, % | 50.8 | | 72 | | 0.01 |
| A pace (%), mean (±SD) | 50 | (30) | 30 | (40) | 0.002 |
| V pace (%), mean (±SD) | 40 | (40) | 60 | (40) | 0.006 |
| FU months, mean (±SD) | 75.5 | (55.5) | 74.3 | (63.2) | 0.5 |
| preSBP (mmHg), mean (±SD) | 130.2 | (9.8) | 121.5 | (6.6) | <0.01 |
| preDBP (mmHg), mean (±SD) | 79 | (5.8) | 79.5 | (7.5) | 0.6 |
| postSBP (mmHg), mean (±SD) | 121.3 | (10.2) | 126.3 | (6.7) | 0.001 |
| postDBP (mmHg), mean (±SD) | 75.9 | (7.5) | 78.1 | (7.3) | 0.09 |
| Pre #meds, mean (±SD) | 3.3 | (0.5) | 3 | (0.1) | <0.001 ^{a} |
| Post #meds, mean (±SD) | 2.7 | (1) | 3.2 | (0.5) | <0.01 ^{b} |
| Pre EF (%), mean (±SD) | 54.6 | (3.6) | 56 | (2.7) | 0.01 |
| Post EF (%), mean (±SD) | 56.1 | (3.1) | 55.8 | (3.6) | 0.7 |

| Medications: | | | |
|---|---|---|---|
| Diuretics, % | 81.7 | 80 | 0.8 |
| BB, % | 57.9 | 68 | 0.2 |
| CCB, % | 61.1 | 30 | <0.01 |
| ACEI/ARB, % | 94.4 | 100 | 0.2 |
| ALPHA, % | 11.9 | 18 | 0.3 |
| ALD, % | 19.8 | 10 | 0.2 |

Responders vs. Non-Responders: Using the described definition, 126 patients were Responders, 50 were Non-Responders. Using the Wald test, the proportion of Responders (126/176) was significantly different from a random 5% occurrence, with a p<0.001.

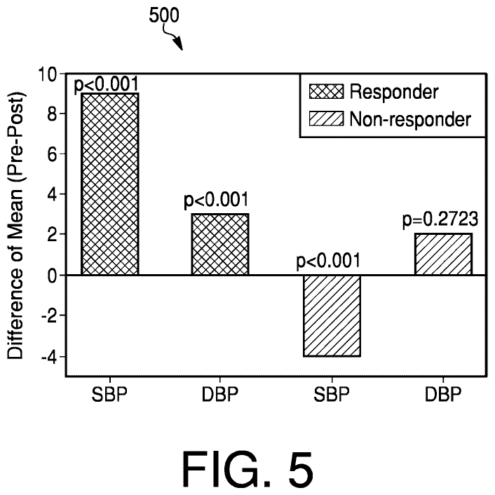

As shown in Table 2. The mean differences in SBP and DBP before and after PP among Responders were highly significant 130±9.8 to 121±10.2 mmHg (SBP), mean drop of9 mmHg, p<0.00 and 79±5.8 to 76±7.5 mHg (DBP), mean drop of 3 mmHg, p<0.001. Conversely, among the Non-Responders, the SBP actually rose 4 mmHg while the D decline of 2 mmHg did not reach statistical significance. The waterfall plot 500 in FIG. 5 graphically displays these data.

TABLE 2

| | | Pre Pacing | | Post Pacing | | Difference | |
|---|---|---|---|---|---|---|---|
| | | Mean (±SD) | | Mean (±SD) | | of Mean (mmHg) | P-value |
| Responders | SBP | 130 | (9.8) | 121 | (10.2) | 9 | <0.01 |
| | DBP | 79 | (5.8) | 76 | (7.5) | 3 | <0.01 |
| Non-Responders | SBP | 122 | (6.6) | 126 | (6.7) | −4 | <0.01 |
| | DBP | 80 | (7.5) | 78 | (7.3) | 2 | 0.3 |

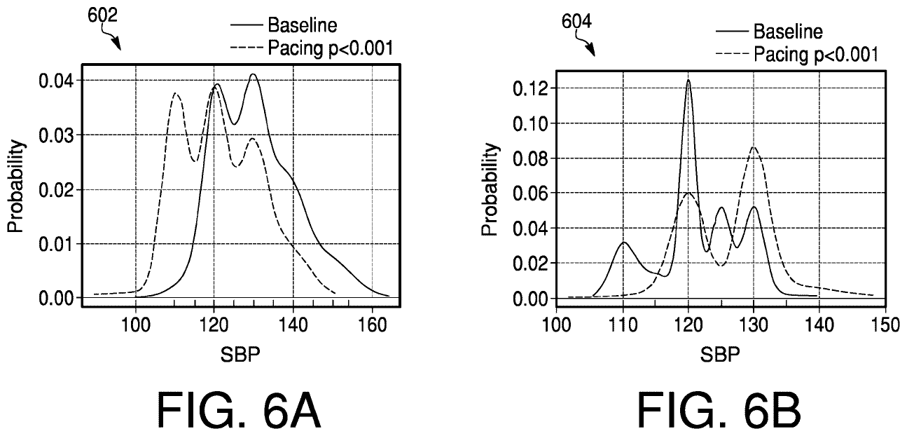

Distribution of systolic blood pressure measurements among Responders: As shown in chart 602 of FIG. 6A and chart 604 of FIG. 61B, the SBP distribution curve among Responders was shown to shift in a statistically significant way to the left, e.g., lower SBP values, following initiation of permanent pacing, with a p<0.001 using the Wilcoxon Signed Rank test.

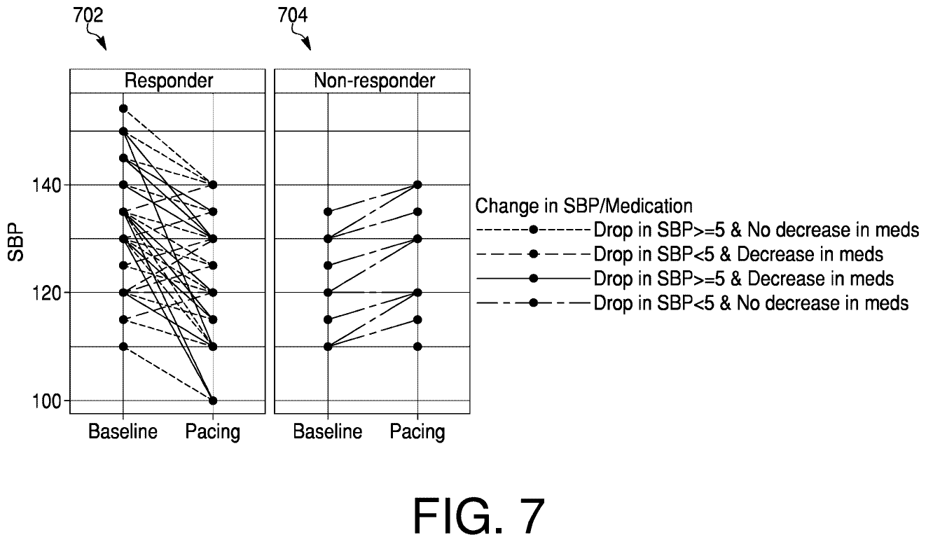

Relationship between response to pacing, decline in SBP and change in number of medications: FIG. 7 graphically displays the relationship between response to PP as it applied to decline in SBP and/or to changes in number of hypertension medications taken. The majority of Responders (as shown by chart 702) had either a decline in SBP≥5 mmHg and a decline in at least one medication, or a decline in SBP without a change in medication. A small number of Responders (n=8) (as shown by chart 704) had a reduction in medication without a decline in SBP of ≥5 mmHg. Among the 50 Non-Responders, 9 patients actually had an increase in the number of medications taken post-PP. The number of lines in the graph in FIG. 7 does not correspond to all the patients, as many patients who had similar responses are grouped into single graph lines for the sake of clarity.

Relationship between the amount of pacing and changes in blood pressure: a statistical correlation was sought between the amount of pacing in each chamber and changes in SBP. Using a linear regression model, correlation between atrial pacing vs. reduction in SBP among the Responders showed a trend towards progressive decline in SBP with increased percentage of atrial pacing, but this did not reach statistical significance (R²=0.022, p=0.09). A trend towards a negative correlation (statistically not significant) was found between right ventricular (RV) pacing and decline in SBP, e.g., fewer changes with increasing RV pacing (Pearson Correlation Coefficient=−0.09).

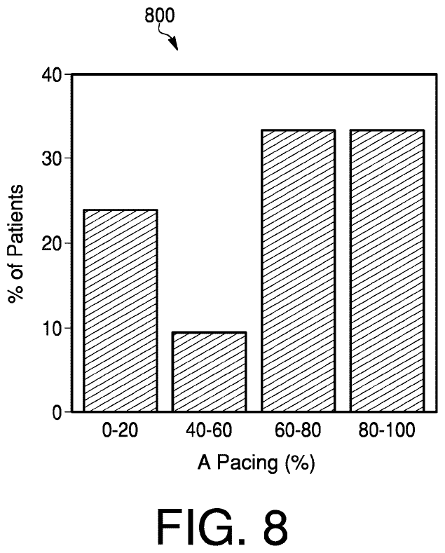

An analysis of Responders who had a decline of >10 mmHg (n=21) showed a stronger correlation with atrial pacing, albeit not yet reaching statistical significance (R²=0.09, p=0.19, Pearson Coefficient=0.3). An exemplary presentation of the data is provided in chart 800 of FIG. 8, wherein patients are presented as bins of percentage of atrial pacing within Responders with a SBP decline of >10 mmHg. As illustrated, most of the patients with this response in SBP were found within the two pacing bins which represent >60% atrial pacing.

Similarly, increased atrial pacing showed a trend toward greater decline in DBP (R²=0.04, p=0.02, correlation=0.2) which did not quite reach statistical significance. Increasing RV pacing was again negatively correlated with decline in DBP (correlation=−0.22).

Association between indication for pacing and BP response: The indication for implantation of permanent dual chamber pacemakers was also examined. Patients were separated into 2 groups: sinus node dysfunction (SSS) vs AV block (AVB), with or without sinus node dysfunction (AVB & SSS+AVB). The rate of Responders among SSS patients (62 of 76 patients, 82%) was significantly different from the rate than among AVB (64 of 100 patients, 64%, p=0.01, Fisher's exact test).

Stratifying blood pressure responses based on percentage paced in each chamber: Based on prior published studies in patients with permanent RV pacing, we stratified the Responders into groups of >40% and <40% RV pacing. Table 3 illustrates the results. An exemplary optimized BP response was seen amongst the 45 patients who paced ≥50% in the right atrium (RA) and <40% in the RV (decline of 12 mmHg in SBP, 6 mmHg in DBP, and 1.62 in number of medications). The results were similar when the RV pacing was dichotomized at 50% (48 patients: reductions of 11, 6 and 1.52 in SBP, DBP and medications, respectively). For the entire study population (n=176), stratification according to atrial and ventricular pacing percentages as above, showed similar trends, with −11 mmHg in SBP, −6 mmHg in DBP, and −1.55 medications in those that were paced >50% in the RA and <40% in the RV.

Changes in BP's and medications stratified by atrial (50%) and ventricular (40%) pacing within responders is shown in Table 3 below:

TABLE 3

| | | | Mean (±SD) | | | | | |
|---|---|---|---|---|---|---|---|---|
| | | | Change in | | Change in | | Change in Number | |
| A Pacing | V Pacing | N | SBP (mmHg) | | DBP (mmHg) | | of Medications | |
| <50% | <40% | 29 | −5.00 | (7.3) | −1.55 | (7.8) | −0.45 | (0.8) |
| <50% | ≥40% | 34 | −9.12 | (5.1) | −3.82 | (8) | 0.21 | (0.5) |
| ≥50% | <40% | 45 | −12.00 | (9.2) | −6.33 | (7.9) | −1.62 | (1) |
| ≥50% | ≥40% | 18 | −7.17 | (2.9) | 3.33 | (6.4) | 0.22 | (0.7) |

In accordance with Table 3, A pacing: atrial pacing; DBP: diastolic blood pressure; SBP: systolic blood pressure; SD: standard deviation; V pacing: ventricular pacing.

Left ventricular ejection fraction: No statistically significant differences were found between the two groups of patients prior to PP. LVEF was statistically not significantly affected by PP in either group (Table 1).

Discussion: The results of this first experiment support and extend prior observations that PP in elderly patients with preserved LVEF and DRH results in statistically significant improvement in BP control. The findings in this study include the following observations: (a) a statistically significant of elderly patients with DRH show a significant decline is SBP following implementation of PP; (b) the magnitude of the improvement in SBP (9 mmHg for the entire Responders group) is significant and encouraging; (c) the improvement in SBP and DBP is notable in the subgroup of patients in whom atrial pacing exceeds 50% and ventricular pacing is less than 40%, where reductions of 12 mmHg and 6 mmHg are seen, respectively; (d) the pacing-related decline in SBP appears to be directly related to the amount of atrial pacing. And while the correlation did not reach statistical significance, most of the Responders who achieved >10 mmHg improvement in SBP were among the group that paced >60% in the atrium; (e) LVEF was similar in both Groups and did not significantly change after pacing in either group.

The effects of neuromodulation on HTN have been the subject of extensive investigation over the past few decades. First studies on baroreflex activation therapy device, endovascular baroreflex amplification therapy, transvenous carotid body and renal artery denervation via catheter ablation have shown favorable results in lowering BP. However, larger, randomized controlled trials may aid in validating invasive procedures on the carotid arteries, and the results of randomized placebo-controlled trials of renal artery denervation have to date shown modest (4/2 mmHg) magnitude of benefit.

Pacemaker-based cardiac neuromodulation therapy (CNT) may be feasible and acceptably safe in patients with HTN and an indication for pacemaker implantation. It should be noted that approximately 66% of the patients who require pacemaker therapy have arterial HTN. The initial unblinded studies of CNT employing a sequence of variably timed short and longer atrioventricular intervals showed a decrease in BP levels in pacemaker patients with DRH. The effects of CNT on BP were confirmed by a double-blind randomized pilot study, and the authors suggested that a combination of decreased ventricular preload and a putative modulation of the autonomic nervous system might prevent baroceptor-based sympathetic activation and serve as potential mechanisms of the observed BP reduction. However, the results of these studies have also raised questions about the long-term safety and quality of life in patients undergoing pacing with short, programmed AV delays. Specifically, chronic RV pacing can increase the risk of heart failure, and long-term pacing with programmed short AV delays can cause ‘pacemaker syndrome’ and various atrial arrhythmias. Both are features of the proposed CNT pacing algorithm. Our study was a retrospective analysis of patients undergoing permanent pacing with conventional clinically indicated programming of dual chamber pacemakers, e.g., minimizing of RV pacing.

This retrospective study, by definition, was not designed to elucidate physiologic mechanisms. However, given the significant changes in BP seen in hypertensive patient population following implantation of permanent pacemakers, two possible physiologic mechanisms are proposed. First, sympathetic nerve activity (SNA) declines with dual chamber pacing among patients with normal LVEF at clinically relevant pacing rates. Intriguingly, the BP lowering effect of increased atrial pacing rate in an office-based pacing study is blunted in patients taking beta-blocking medications, suggesting a blockade of pacing-induced lowering of SNA. Further insight might be provided by a study in which ambulatory SNA is measured in a similar patient population.

Secondly, atrial pacing in an experimental animal model is associated with a significant release of endogenous atrial natriuretic peptide, ANP. While BNP levels have generally thought to indicate worsening of LV function, hence elevated levels are associated with RV pacing and lessened by dual chamber pacing, nonetheless, it may be the case that the increased atrial pacing seen in this study's Responders may have engendered BNP release which may have played a role in the improved control of BP in these patients.

Limitations: The main limitation of the present study is its retrospective and non-randomized design. However, the relatively long follow up period (mean of >6 years), the relatively large number of patients and the conclusive nature of the data are the strengths of this study. The known acceptable safety profile of permanent pacing in hypertensive patients and the strong efficacy observed in the present study support further prospective randomized studies.

Conclusions: This relatively large retrospective long-term study provides initial evidence that HTN treatment with a conventional dual chamber pacemaker-based device appears safe and effective at intermediate and long-term follow-up. The current study included patients with DRH who have standard indications for PP. The significant reduction in SBP and DBP is more closely related to atrial pacing, in contrast to studies in patients with DRH and permanent pacing, as discussed. If validated, a clear advantage of using BP as an end point for adjusting atrial pacing becomes self-evident. Factors requiring further clarification in longer-term randomized studies include assessments of safety (impact on LV size and function, atrial size, and arrhythmias), and the impact on BNP, and autonomic nervous system activity. With further proof of safety and efficacy, pacing therapy for hypertension may yet be a new indication for PP in the appropriate patient population.

Clinical Perspectives: Drug-resistant hypertension is a major global epidemiologic problem. Novel approaches to the treatment are being developed and include possible device-based treatment of hypertension. This experiment suggests that hypertension can be ameliorated by titrating the atrial pacing rate of permanent pacemakers.

### Experiment 2: Safety and Efficacy of Adaptive Atrial Pacing Regulated by Blood Pressure During Low-Level Exercise

The following disclosure corresponds to a second experiment conducted in accordance with the subject matter discussed herein. Introduction: Heart failure patients with preserved ejection fraction (HFpEF) comprise half of all patients with heart failure. Patients with HFpEF present with exercise intolerance and diminished increases in heart rate (HR) with exercise. Chronotropic incompetence is present in over 50% of these patients.

Despite the prevalence of HFpEF, treatment options are limited. Current implantable cardiac pacemakers augment cardiac output by increasing heart rate (HR) in response to increased cardiac demand using activity-based rate adaptive atrial pacing (DDDR); however, a recent study investigating modified DDDR programming where the programmed HR and slope were increased for both daily living and peak exercise. Despite the increased HR these patients failed to demonstrate improvement in exercise performance or quality of life.

In this second experiment, the results of a small proof of concept study at two large cardiology clinics comparing two pacing modalities by measuring exercise duration of HFpEF patients undergoing treadmill tests are reported. The first pacing modality consisted of subjects' standard pacemaker programming (DDD or DDDR), and the second, a simulation of a pacing algorithm, which modulates atrial pacing rate based on blood pressure (BP) measurements (BPAP).

Methods: Subjects underwent two modified Bruce protocol graded treadmill exercise tests in which pacemaker programming was randomized to either standard programming, or BPAP at least one week apart. A 30-minute rest period occurred immediately prior to and post exercise. Physiological measurements of HR, and systolic and diastolic blood pressure (SBP and DBP) were collected throughout.

During the BPAP treadmill test, the pacemaker activity sensor was disabled and the pacing algorithm instructed the pacemaker technician to increase, decrease, or leave unchanged the atrial pacing rate based on BP measurements obtained in two-minute increments. Post treadmill test, pacemaker programming was restored to original settings.

Subjects and clinical staff were blinded to pacemaker programming, the pacemaker technician was unblinded. Institutional Review Board approval was obtained prior to study initiation. Informed consent was obtained prior to any study related activity.

Results: Clinical Characteristics: Ten subjects with HFpEF associated with hypertension who also had permanent dual chamber pacemakers, previously implanted for standard clinical indications, participated in the study. Mean age was 70.1±6.8, left ventricular ejection fraction of 54.8±1.9%, 50% male, and 90% Caucasian. Sixty percent of subjects had been programmed to DDDR with the remainder programmed to DDD. Subjects had an average resting HR of 65.5±8.7-bpm, and SBP/DBP of 143/83-mmHg. All patients were taking beta-blockers at the time of enrollment.

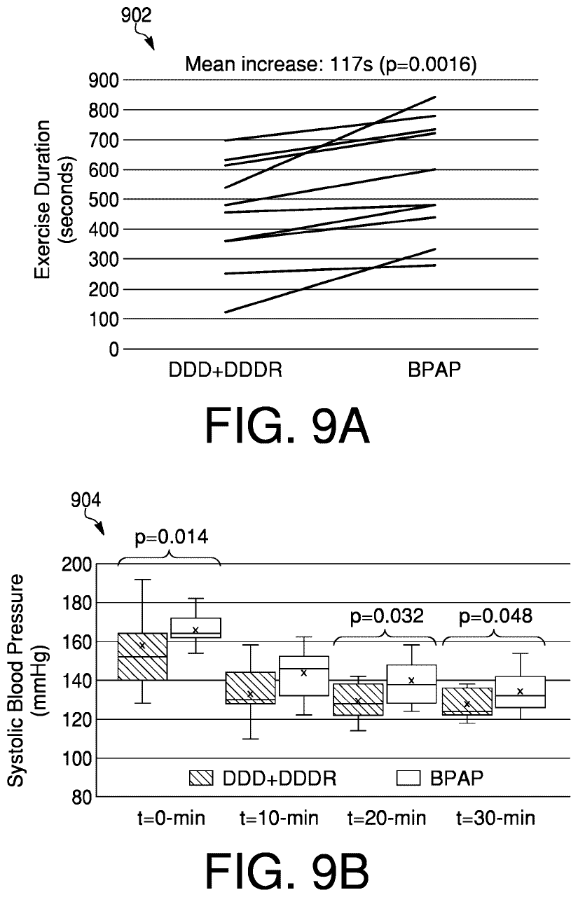

Treadmill and Recovery Period Results: Exercise duration increased in all 10 subjects, when paced in the BPAP mode as, shown at chart 902 of FIG. 9A, compared to standard pacemaker programming. A mean increase of 117 s (26%, p=0.0016) which ranged from 22 to 300 s (5 to 164%). Improvements were similar for subjects programmed to either DDD or DDDR.

In the post-treadmill recovery period, SBP was higher for subjects who underwent BPAP, as shown at chart 904 of FIG. 9B. This difference in SBP was most pronounced immediately post-treadmill (t=0-minutes) and diminished as subjects progressed through the recovery period (t=30-minutes). Statistical significance was achieved at t=0, 20, and 30-minutes post-treadmill.

Safety Profile: No adverse events including palpitations, chest pain, or dizziness were reported for either pacing modality showing that BPAP is at least as acutely safe as standard programming.

Discussion: An increase in exercise capacity has been reported in HFpEF patients using a pacemaker responding to a physiologic input, namely, BP. All subjects experienced an increase in exercise duration utilizing BPAP compared to standard programming. These results demonstrate that simply increasing HR based on activity level is not effective and a more physiological parameter modulates HR.

Modulation of HR should utilize a physiologically based proxy with a similar time course as the sinus node. Blood pressure fits these requirements and is inherently dynamic and diurnal, further highlighting its potential superiority in modulating HR response to exercise.

In this study, there were 44 opportunities where the pacing algorithm discussed herein instructed the pacemaker technician to increase, decrease, or leave unchanged the atrial pacing rate based on BP measurements. This suggests that a dynamic closed-loop system utilizing BPAP may have a salutary effect in patients with HFpEF.

Statistically significant differences in SBP observed during the post exercise period may yield clues to the mechanisms underlying the increase in exercise duration. Additionally, anecdotal reports from each of the ten subjects obtained after the study indicated a strong preference for how each felt while exercising when using BPAP, compared to subjective response to exercise with standard device programming.

Conclusions: The promising results presented here warrant further investigation of BPAP to further characterize the safety, efficacy and possible mechanisms in the acute and longer-term treatment in this patient group.

Based on successful experience in treating patients suffering from hypertension with blood-pressure based ‘closed loop’ atrial pacing, wherein the pacing rate was adjusted in response to blood pressure, a proof-of-concept experiment in 10 patients with known HFpEF and concomitant hypertension is performed, with previously implanted pacemaker systems. The experiment reported herein is an office based randomized study in which each patient underwent two, low-level treadmill exercise tests in a randomized sequence. The statistically significant results showed longer exercise time when pacing was adjusted based on measured BP during the treadmill exercise test compared to standard pacemaker programming (either DDD or DDDR). Conceivably, a new paradigm in pacemaker management of patients with HFpEF might emerge from this line of research.

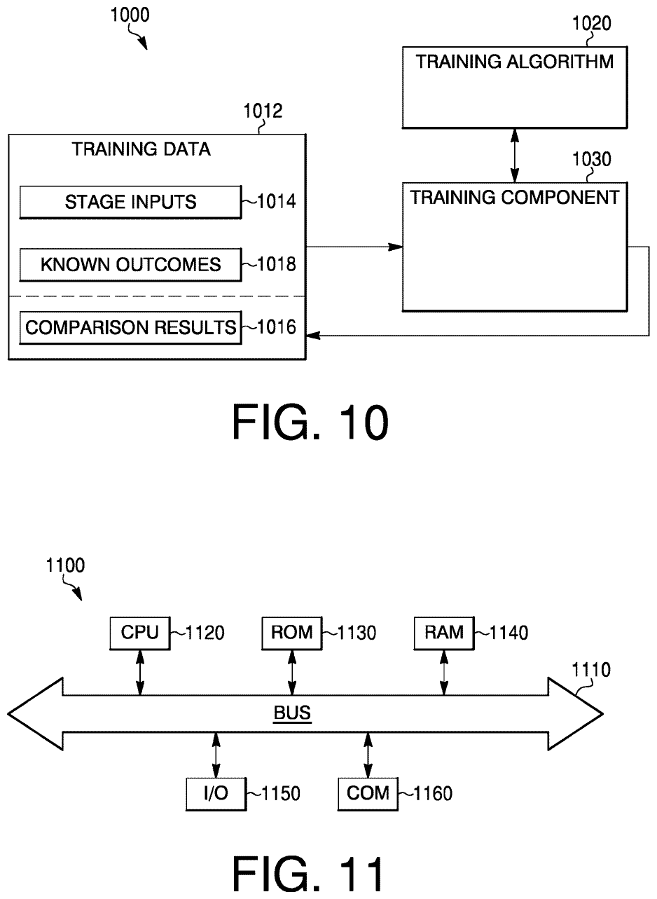

One or more implementations disclosed herein may be applied by using a machine learning model, another AI system such as a neural network, or a non-AI rules-based system. For example, a machine learning model may be used to determine a state machine and/or a next state. As shown in flow diagram 1000 of FIG. 10, training data 1012 may include one or more of stage inputs 1014 and known outcomes 1018 related to a machine learning model to be trained. The stage inputs 1014 may be from any applicable source including an input or system discussed herein (e.g., an output from a step or aspect of FIGS. 1-98). The known outcomes 1018 may be included for machine learning models generated based on supervised or semi-supervised training. An unsupervised machine learning model might not be trained using known outcomes 1018. Known outcomes 1018 may include known or desired outputs for future inputs similar to or in the same category as stage inputs 1014 that do not have corresponding known outputs.

The training data 1012 and a training algorithm 1020 may be provided to a training component 1030 that may apply the training data 1012 to the training algorithm 1020 to generate a machine learning model. According to an implementation, the training component 1030 may be provided comparison results 1016 that compare a previous output of the corresponding machine learning model to apply the previous result to re-train the machine learning model. The comparison results 1016 may be used by the training component 1030 to update the corresponding machine learning model. The training algorithm 1020 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as the flows and/or process discussed herein (e.g., in FIGS. 1-9B), etc., may be performed by one or more processors of a computer system, such any systems or devices used to implement the techniques disclosed herein. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

FIG. 11 depicts an example system 1100 that may execute techniques presented herein. FIG. 11 is a simplified functional block diagram of a computer that may be configured to execute techniques described herein, according to exemplary embodiments of the present disclosure. Specifically, the computer (or “platform” as it may not be a single physical computer infrastructure) may include a data communication interface 1160 for packet data communication. The platform may also include a central processing unit (“CPU”) 1120, in the form of one or more processors, for executing program instructions. The platform may include an internal communication bus 1110, and the platform may also include a program storage and/or a data storage for various data files to be processed and/or communicated by the platform such as ROM 1130 and RAM 1140, although the system 1100 may receive programming and data via network communications. The system 1100 also may include input and output ports 1150 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”). Wide Area Network (“WAN”), and/or the Internet. Similarly, techniques presented herein as involving multiple devices may be implemented in a single device. In a distributed computing environment, program modules may be located in both local and/or remote memory storage devices.

As discussed herein, a memory may include a device or system that is used to store information for immediate use in a computer or related computer hardware and digital electronic devices. Contents of memory can be transferred to storage (e.g., via virtual memory). Memory may be implemented as semiconductor memory, where data is stored within memory cells built from MOS transistors on an integrated circuit. Semiconductor memory may include volatile and/or non-volatile memory. Examples of non-volatile memory include flash memory and read-only memory (ROM), programmable read-only memory (PROM), erasable programmable read-only memory (EPROM), electrically erasable programmable read-only memory (EEPROM) memory, and the like. Examples of volatile memory include primary memory such as dynamic random-access memory (DRAM) and fast CPU cache memory such as static random-access memory (SRAM).

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

It should be understood that embodiments in this disclosure are exemplary only, and that other embodiments may include various combinations of features from other embodiments, as well as additional or fewer features. It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A method for outputting a pacing rate, the method comprising:
receiving a heart rate and a blood pressure of a patient;
determining a first pacing rate to modify the heart rate based on the heart rate and the blood pressure;
outputting the first pacing rate;
receiving a subjective input subsequent to outputting the first pacing rate;
determining a second pacing rate based on the subjective input and the first pacing rate; and
outputting the second pacing rate.

2. The method of claim 1, wherein determining the first pacing rate to modify the heart rate is further based on at least one of physiological parameters, environmental parameters, and/or emotional parameters.

3. The method of claim 2, wherein the physiological parameters include one or more of the patient's current heart rate, current blood pressure, temperature, medication compliance, blood oxygen level, glucose level, blood electrolytes level, an accelerometer value, a respiratory rate sensor value, a thoracic impedance, an impedance, portions of cardiac rate such as atrial rate, ventricular rate, atrioventricular conduction, rhythm irregularities, autonomic nervous system (ANS) function, glucose, skin electrolytes, galvanic skin response, Photoplethysmography (PPG) values, electroencephalogram (EEG) wave, and/or urination parameters.

4. The method of claim 2, wherein the environmental parameters include one or more of a patient diet, a time of day, an ambient temperature, a patient location, an ambient oxygen concentration, or a humidity.

5. The method of claim 1, wherein the first pacing rate or the second pacing rate is output by a pacing machine learning algorithm.

6. The method of claim 1, wherein the blood pressure is systolic or diastolic.

7. The method of claim 1, further comprising:
determining whether the blood pressure of the patient is one of above a first threshold blood pressure or below a second threshold blood pressure; and
determining the first pacing rate further based on whether the blood pressure of the patient is one of above the first threshold blood pressure or below the second threshold blood pressure.

8. The method of claim 7, wherein the first pacing rate is one of higher than the heart rate by a first pacing value if the blood pressure of the patient exceeds the first threshold blood pressure or is lower than the heart rate by a second pacing value different than the first pacing value, if the blood pressure of the patient is below the second threshold blood pressure.

9. The method of claim 1, wherein upon expiration of a predetermined time period or a dynamically determined time period:
receiving an updated heart rate and an updated blood pressure of the patient;
determining a third pacing rate to modify the updated heart rate based on the updated heart rate and the updated blood pressure;
outputting the third pacing rate;
receiving an updated subjective input subsequent to outputting the third pacing rate;
determining a fourth pacing rate based on the updated subjective input and the third pacing rate; and
outputting the fourth pacing rate.

10. The method of claim 1, wherein the first and second pacing rates are further determined based on patient attributes.

11. The method of claim 1, wherein receiving the subjective input comprises:
receiving a sensor input from a physiological sensor;
providing the sensor input to a pseudo-subjective machine learning model trained to generate outputs based on at least one of historical sensor inputs, simulated sensor inputs, historical subjective inputs, or simulated subjective inputs; and
receiving, as an output from the pseudo-subjective machine learning model, the subjective input.

12. The method of claim 1, wherein the first pacing rate is further based on one of a blood pressure device, a pacing device, or a subjective input device.

13. A system for outputting a pacing rate, the system comprising:
a processor; and
a memory having programming instructions stored thereon, which, when executed by the processor, causes the system to perform operations comprising:
receiving a heart rate and a blood pressure of a patient;
determining a first pacing rate to modify the heart rate based on the heart rate and the blood pressure;
outputting the first pacing rate;
receiving a subjective input subsequent to outputting the first pacing rate;
determining a second pacing rate based on the subjective input; and
outputting the second pacing rate.

14. The system of claim 13, wherein determining the first pacing rate to modify the heart rate is further based on at least one of physiological, environmental, and/or emotional parameters.

15. The system of claim 13, wherein the operations further comprise:
determining whether the blood pressure of the patient is one of above a first threshold blood pressure or below a second threshold blood pressure;
determining the first pacing rate further based on whether the blood pressure of the patient is one of above the first threshold blood pressure or below the second threshold blood pressure.

16. The system of claim 13, wherein receiving the subjective input comprises:
receiving a sensor input from a physiological sensor;
providing the sensor input to a pseudo-subjective machine learning model trained to output the subjective input based on at least one of historical sensor inputs, simulated sensor inputs, historical subjective inputs, or simulated subjective inputs; and
receiving, from the pseudo-subjective machine learning model, the subjective input.

17. A non-transitory medium for outputting a pacing rate and having a sequence of instructions, which, when executed by a processor, causes a computing system to perform operations comprising:
receiving a heart rate and a blood pressure of a patient;
determining a first pacing rate to modify the heart rate based on the heart rate and the blood pressure;
outputting the first pacing rate;
receiving a subjective input subsequent to outputting the first pacing rate;
determining a second pacing rate based on the subjective input; and
outputting the second pacing rate.

18. The non-transitory medium of claim 17, wherein determining the first pacing rate to modify the heart rate is further based on at least one of physiological, environmental, and/or emotional parameters.

19. The non-transitory medium of claim 17, further comprising:
determining whether the blood pressure of the patient is one of above a first threshold blood pressure or below a second threshold blood pressure; and
determining the first pacing rate further based on whether the blood pressure of the patient is one of above the first threshold blood pressure or below the second threshold blood pressure.

20. The non-transitory medium of claim 17, wherein receiving the subjective input comprises:
receiving a sensor input from a physiological sensor;
providing the sensor input to a pseudo-subjective machine learning model trained to output the subjective input based on at least one of historical sensor inputs, simulated sensor inputs, historical subjective inputs, or simulated subjective inputs; and
receiving, from the pseudo-subjective machine learning model, the subjective input.
